(19) United States
(12) Patent Application Publication
(10) Pub. No.: US 2009/0076316 A1
(43) Pub. Date: Mar. 19, 2009

(54) PROCESS FOR PRODUCTION OF AROMATIC HYDROCARBON

(75) Inventors: Masaru Ichikawa, Tokyo (JP); Ryoichi Kojima, Saitama (JP); Satoshi Kikuchi, Mie (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: Masaru ICHIKAWA, Suginami-ku, Tokyo (JP)

(21) Appl. No.: 11/816,068

(22) PCT Filed: Feb. 8, 2006
(86) PCT No.: PCT/JP2006/302178

§ 371 Date: Aug. 10, 2007

(30) Foreign Application Priority Data
Feb. 10, 2005 (JP) 2005-068051
Jul. 19, 2005 (JP) 2005-208437

(51) Int. Cl. C07C 2/52

(52) U.S. Cl. 585/419; 502/158

(57) ABSTRACT

A process for producing an aromatic hydrocarbon, comprises heating the hydrocarbon in the presence of a catalyst carrying a molybdenum compound or a rhenium compound on a metallosilicate carrier modified with a silicon compound, a sodium compound or a calcium compound. The silicon compound is a silane compound having a basic group selected from amino, alkylamino and pyridyl groups and an organic group of a size equal to or greater than the pore size of the metallosilicate and selected from trialkoxy and triphenyl groups and the sodium compound or the calcium compound is a compound having an organic group of a size equal to or greater than the pore size of the metallosilicate and selected from crown ether, hexafluoropentanedione and acetylacetonate. The silane compound, the sodium compound or the calcium compound is modified so as to make an oxide thereof by impregnating the metallosilicate carrier with it and subsequently heat-treating it in an oxygen-containing atmosphere.

### TECHNICAL FIELD

The present invention relates to a process for producing an aromatic hydrocarbon such as benzene from a lower hydrocarbon such as methane. More particularly, the present invention relates to a process for producing an aromatic hydrocarbon, using a hydrocarbon having 1 to 8 carbon atoms obtained from gas such as liquid petroleum gas, liquid natural gas, coal carbonization gas, refined petroleum gas, naphtha, organic matter fermentation gas, organic matter dry distilled gas, coal reformed gas, methane hydrate recycling gas or a decomposition product of any of such gases as starting material. The present invention also relates to a catalyst for producing an aromatic hydrocarbon, using a lower hydrocarbon as starting material.

### BACKGROUND ART

While aromatic hydrocarbons such as benzene, toluene and xylene have been and being mainly produced from naphtha that originates from petroleum, processes for producing an aromatic hydrocarbon such as methane or benzene from a lower hydrocarbon directly by a catalytic reaction in the presence of a catalyst, which is molybdenum carried on synthetic zeolite ZSM-5, are known (see, inter alia, Non-Patent Document 1). However, the use of such a catalyst is accompanied by technological problems including an increased deposition of carbon, a low conversion ratio of methane and a rapid degradation of catalyst activity.

Processes for producing an aromatic hydrocarbon from a material gas such as methane or methane-containing lower hydrocarbons directly by a catalytic reaction in the presence of a catalyst of molybdenum or zinc carried on ZSM-5 with or without carbon dioxide added to the material gas by a small amount have been proposed (see, inter alia, Patent Documents 1 and 2).

With any of these processes, it has been confirmed that an aromatic compound such as benzene is produced efficiently from methane-containing lower hydrocarbons and the catalyst performs excellently for a long time. However, such improved processes for converting methane into an aromatic hydrocarbon, using a catalyst carried on ZMS-5, are still accompanied by problems such as that aromatic hydrocarbons including alkyl benzenes and naphthalene are produced with a selectivity of not lower than 20% to reduce the selectivity of benzene to about 70% and that the performance of the catalyst is degraded to a certain extent as the reaction time increases, which make the processes less feasible.

Thus, there is a strong demand for catalysts that show an excellent reaction efficiency and also can raise the aromatic hydrocarbon production efficiency and the benzene selectivity and stably maintain the catalyst performance for a long time.

### Non-Patent Document 1: JOURNAL OF CATALYSIS, 165(2),150-161 (1997)

### Patent Document 1: JP-A-10-272366

### Patent Document 2: JP-A-11-60514

### DISCLOSURE OF THE INVENTION

### Problem to be Solved by the Invention

Therefore, it is the problem to be solved by the present invention to provide a catalyst for simultaneously producing benzene or aromatic compounds containing benzene as main component and hydrogen gas with a high conversion ratio and a high selectivity, using a lower hydrocarbon such as methane as starting material, that can stably maintain its catalyzing ability of converting a lower hydrocarbon into an aromatic compound for a long time and also a process for producing benzene or aromatic compounds containing benzene as main component and hydrogen from a lower hydrocarbon by using such a catalyst.

### Means for Solving the Problem

According to the present invention, the above problem of the invention can be dissolved by providing a process for producing an aromatic hydrocarbon, using a lower hydrocarbon as starting material, the process comprising heating the hydrocarbon in the presence of a catalyst carrying a molybdenum compound or a rhenium compound on a metallosilicate carrier modified with a silicon compound, a sodium compound or a calcium compound, characterized in that the silicon compound is a silane compound having a basic group selected from amino, alkylamino and pyridyl groups and an organic group of a size equal to or greater than the pore size of the metallosilicate and selected from trialkoxy and triphenyl groups and the sodium compound or the calcium compound is a compound having an organic group of a size equal to or greater than the pore size of the metallosilicate and selected from crown ether, hexafluoropentanedione and acetylacetonate, the silane compound, the sodium compound or the calcium compound being modified so as to make an oxide thereof by impregnating the metallosilicate carrier with it and subsequently heat-treating it in an oxygen-containing atmosphere.

Preferably, a process for producing an aromatic hydrocarbon as defined above is characterized in that the catalyst contains platinum or rhodium.

In another aspect of the present invention, there is provided a catalyst for producing an aromatic hydrocarbon, using a lower hydrocarbon as starting material, the catalyst carrying a molybdenum compound or a rhenium compound on a metallosilicate carrier modified with a silicon compound, a sodium compound or a calcium compound, characterized in that the silicon compound is a silane compound having a basic group selected from amino, alkylamino and pyridyl groups and an organic group of a size equal to or greater than the pore size of the metallosilicate and selected from trialkoxy and triphenyl groups and the sodium compound or the calcium compound is a compound having an organic group of a size equal to or greater than the pore size of the metallosilicate and selected from crown ether, hexafluoropentanedione and acetylacetonate, the silane compound, the sodium compound or the calcium compound being modified so as to make an oxide thereof by impregnating the metallosilicate carrier with it and subsequently heat-treating it in an oxygen-containing atmosphere.

Preferably, a catalyst for producing an aromatic hydrocarbon as defined above is characterized by carrying platinum or rhodium compound in addition to the molybdenum compound or the rhenium compound.

### ADVANTAGES OF THE INVENTION

A process for producing an aromatic hydrocarbon according to the present invention can produce an aromatic hydrocarbon from a lower hydrocarbon such as methane with a high conversion ratio and a high selectivity. Particularly, it provides a high benzene selectivity and hence can stably produce aromatic hydrocarbons containing benzene as main component for a long time.

### BEST MODE FOR CARRYING OUT THE INVENTION

As a result of research efforts, the inventor of the present invention found that a catalyst having a metallosilicate carrier that is modified typically by means of aluminum silicate to produce aromatic hydrocarbons can improve the conversion ratio of aromatic hydrocarbons containing benzene as main component, reduce the ratio of naphthalene in the conversion product and stably show a good performance for a long time if compared with conventional catalysts carrying a metal component such as molybdenum on a metallosilicate carrier.

A metallosilicate to be used as catalyst carrier for producing an aromatic hydrocarbon is a porous substance referred to as zeolite that contains silica and alumina as main components and can be selected from molecular sieve5A, faujasite (NaY) and NaX, ZSM-5, ZSM-11, ZRP-1, MCM-22, ferriorite and β-zeolite. Preferably, the metallosilicate has a pore diameter between 0.4 and 0.7 nm.

For a reaction of converting a lower hydrocarbon into an aromatic compound with a conversion ratio and a selectivity that are feasible for practical applications according to the present invention, the silica/alumina ratio is preferably between 10 and 100, more preferably between 20 and 70.

The metallosilicate carrier of a catalyst according to the present invention is chemically modified by a silicon compound, a sodium compound, a calcium compound or the like.

Silicon compounds that can be used for modifying the metallosilicate carrier include those having a basic group that reacts with the metallosilicate surface such as an amino group, an alkylamino group or a pyridyl group and a bulky organic group of a size equal to or greater than the pore size of the metallosilicate such as a trialkoxy group or a triphenyl group.

Specific examples of silicon compound that can be used for the purpose of the present invention include methoxyethyltriethoxysilane, 3-acetoxypropyltriethoxysilane, 3-aminopropylethoxysilane, N-(2-aminoethyl)-3-aminopropyltrimethoxysilane, aminophenyltrimethoxysilane, 3-aminopropyldimethylethoxysilane, 3-aminopropyltrimethoxysilane, N-phenylaminopropyltriethoxysilane, 2-(4-pyridyl) triethoxysilane, N-(3-triethoxysilylpropyl)-4,5, dihydroimidazole, 2-(triethoxysilyl)pyridine, 3-(trimethoxysilylpropyl) diethylenetriamine, bis(trimethoxylpropyl)amine, bis(methyldiethoxypropyl)amine, bis-chloromethyldichlorosilane, 3-bromopropyltrichlorosilane, 3-bromopropyltrimethoxysilane, parachloromethyltrimethoxysilane, chloromethyltriethoxysilane, cyclopentylsilane, diethylsilane, dimethylchlorosilane, dimethyldichlorosilane, dimethyldiethoxysilane, dimethylethoxysilane, ethylmethoxysilane, phenyltrimethoxysilane, triphenylaminosilane, triphenylsilane, tetraethoxysilane, dichlorosilane, diiodosilane, silane and trimethylsilane.

More preferably, the silicon compound is selected from methoxyethyltriethoxysilane, 3-acetoxypropylethoxysilane, 3-aminopropylethoxysilane, N-(2-aminoethyl)-3-aminopropyltrimethoxysilane, aminophenyltrimethoxysilane, 3-aminopropyldimethylethoxysilane, 3-aminopropyltrimethoxysilane, N-phenylaminopropyltriethoxysilane, 2-(4-pyridyl)triethoxysilane, N-(3-triethoxysilylpropyl)-4,5, dihydroimidazole, 2-(triethoxysilyl)pyridine, 3-(trimethoxysilylpropyl) diethylenetriamine, bis(trimethoxypropyl) amine and bis(methyldiethoxypropyl)amine.

Techniques for causing a metallosilicate to carry a silicon compound include impregnating the metallosilicate carrier with an organic solvent solution containing a silicon compound such as a solution using benzene, toluene, methylene chloride, chloroform, hexane, tetrafuran, ethanol, propyl alcohol, diethyl ether or the like as solvent or causing the metallosilicate to adsorb and carry a silicon compound in an inert gas atmosphere such as nitrogen, helium or argon or in an hydrogen or carbon dioxide atmosphere and subsequently heat-treating the metallosilicate in an oxygen-containing atmosphere.

For example, a catalyst can be prepared for the purpose of the present invention by impregnating powdery ZSM-5 with a toluene solution of 3-aminopropyltriethoxysilane, removing the solvent by distillation under reduced pressure and subsequently heat-treating the impregnated powdery ZSM-5 at 250 to 800° C., preferably at 350 to 600° C., in an air flow to chemically modify the ZMS-5.

When causing a metallosilicate carrier to carry a silicon compound, the ratio of the silicon compound relative to the metallosilicate carrier is preferably 0.001 to 50 weight portions, more preferably 0.01 to 10 weight portions, most preferably 0-1 to 5 weight portions, relative to 100 weight portions of the metallosilicate in terms of silicon oxide (SiO₂).

When a sodium compound or a calcium compound is carried by a chemically modified metallosilicate, the sodium compound or the calcium compound is preferably a sodium complex or a calcium complex, whichever appropriate, having a bulky organic group. Examples of such compounds include 12-crown-4 (1,4,7,10-tetraoxacyclododecane), 15-crown-5 (1,4,7,10,13-pentaoxacyclododecane), 18-crown-6 (1,4,7,10,13,16-hexaoxacyclooctadecane, (18-crown-6)-2,3,11,12-tetracarboxylic acid, 2-aminomethyl-15-crown-5,2-(hydroxymethyl)-12-crown-4,2-(hydroxymethyl)-15-crown-5,2-(hydroxymethyl)-18-crown-6,2-(hydroxymethyl) anthraquinone, hexafluoropentadionesodium, sodium 2,4-pentadione salts, lithiumtetramethylpentanedione salts, trimethylsilanate sodium salts, N-(trimethoxysilylpropyl)diethylenetriamine, t-butoxysodium, Na (Li, K) phthalocyanine, tri-t-butylpropylalcoxide, calcium acetylacetate, calcium-bis(6,6,7,7,8,8,8) heptafluoro-2,2-dimethyl-3,5-octadione, calcium hexafluoroacetylacetate and cyclic ethylenediamino compounds.

Techniques that can be used for modifying the metallosilicate by means of an organic compound containing either of the above metals are similar to those listed above for modifying the metallosilicate by means of a silicon compound.

A catalyst according to the present invention can be manufactured by causing a metallosilicate carrier modified by means of a silicon compound, a sodium compound or a calcium compound in the above-described manner to carry at least molybdenum or rhenium. More specifically, the metallosilicate carrier can be made to carry molybdenum or rhenium by impregnating the metallosilicate with a solution of a precursor of the metal component and heating the impregnated metallosilicate in an oxygen-containing atmosphere.

Examples of precursors of molybdenum and rhenium that can be used for the purpose of the present invention include ammonium paramolybdate, phosphomolybdic acid and 12-silico-molybdic acid as well as oxides, halides such as chlorides and bromides, salts of mineral acids such as nitrates, sulfates and phosphates, carboxylic salts such as carbonates, acetates and oxalates, carbonyl complexes and metal complexes including complexes of metal salts such as acetylacetates of molybdenum and rhenium.

The chemically modified metallosilicate carrier can be made to carry a molybdenum compound or a rhenium compound by impregnating it with an aqueous solution of the compound or by means of a ion-exchange process and heat-treating it in air.

For instance, a chemically modified metallosilicate catalyst carrying molybdenum can be prepared by impregnating the chemically modified metallosilicate carrier with an aqueous solution of ammonium molybdate, drying it and subsequently heat-treating it at 250 to 800° C., preferably at 350 to 600° C. in an air flow.

When causing a chemically modified metallosilicate carrier to carry molybdenum or rhenium, for example, the mass ratio of the molybdenum to the chemically modified metallosilicate is preferably 0.001 to 50 weight portions, more preferably 0.01 to 30 weight portions, relative to 100 portions of chemically modified metallosilicate.

A catalyst according to the present invention may further contain platinum or rhodium in addition to the molybdenum or rhenium component.

When a platinum or rhodium component is introduced into a catalyst according to the present invention, a compound selected from compounds containing halides such as chlorides and bromides, nitrates, sulfates, phosphates and carboxylic salts such as carbonates, acetates and oxalates of them can be used as starting material.

The mass ratio of the platinum or the rhodium relative to the carrier is preferably 0.001 to 50 weight portions, more preferably 0.01 to 40 weight portions, relative to 100 portions of the carrier.

As in the case of introducing molybdenum into the chemically modified metallosilicate carrier, the chemically modified metallosilicate carrier can be made to carry platinum or rhodium by impregnating the chemically modified metallosilicate carrier with an aqueous solution of a precursor of platinum or rhodium and subsequently heating the impregnated metallosilicate carrier in air.

Techniques that can be used to cause the carrier of the catalyst to carry the metal components include (1) causing the carrier to carry at least either the molybdenum component or the rhenium component and subsequently carry at least either platimum or rhodium component, (2) causing the carrier to carry at least either platinum or rhodium component and subsequently carry either the molybdenum component or the rhenium component and (3) causing the carrier to carry the metal components by means of a solution containing at least either the molybdenum component or the rhenium component and at least either the platinum component or the rhodium component.

With an example of such a method for causing the carrier to carry the metal components, a chemically modified metallosilicate carrier is impregnated with a predetermined quantity of an aqueous solution of ammonium molybdate, then dried, subsequently heat-treated at 250 to 800° C., preferably at 350 to 600° C. in an air flow, thereafter impregnated with an aqueous solution of chloroplatinic acid or rhodium chloride, then dried, and finally heat-treated at 250 to 800° C., preferably at 350 to 600° C. in an air flow to produce a chemically modified metallosilicate catalyst carrying a molybdenum component and a platinum component or a chemically modified metallosilicate catalyst carrying a molybdenum component and a rhodium component.

While the chemically modified metallosilicate carrier is impregnated with a compound containing the metal component of the catalyst to carry the latter and subsequently heat-treated in the above description, alternatively the metallosilicate carrier may be impregnated with an aqueous solution of ammonium molybdate to carry the latter, dried, then heat-treated at 250 to 800° C., preferably at 350 to 600° C., in an air flow, additionally impregnated with an aqueous solution of chloroplatinic acid or rhodium chloride, dried, then heated at 250 to 800° C., preferably at 350 to 600° C., in an air flow so as to carry a molybdenum component and a platinum component or a molybdenum component and a rhodium component, subsequently impregnated with an organic solvent solution of toluene containing 3-aminopropyltriethoxysilane to carry the latter, removing the solvent under reduced pressure and then heat-treated at 250 to 800° C., preferably at 350 to 600° C., in an air flow to produce a catalyst according to the present invention.

In a catalyst for producing an aromatic hydrocarbon according to the present invention, it is safe to assume that molybdenum or rhenium and platinum or rhodium exists therein in the form of metal or metal oxide. Therefore, for the purpose of the present invention, the expression of the molybdenum component in a catalyst according to the present invention refers to that the catalyst contains molybdenum at least in the form of metal or in the form of metal oxide.

With a process for producing an aromatic hydrocarbon according to the present invention, an aromatic hydrocarbon is produced by way of a catalytic reaction, using a lower hydrocarbon as starting material and a catalyst having a chemically modified metallosilicate carrier as described above that is made to carry a molybdenum component or a rhenium component with or without a platinum component or a rhodium component added thereto, in a gas phase where oxygen does not exist at 300 to 800° C., preferably at 450 to 775° C., under pressure of 0.01 to 1 MPa, preferably of 0.1 to 0.7 MPa.

The reaction may be conducted in the form of batch reaction or flow reaction, although it is preferable to conduct the reaction in the form of flow reaction using a fixed bed, a moving bed or fluidized bed.

When the form of flow reaction is employed, the weight hourly space velocity (WHSV) is 0.1 to 10, preferably 0.5 to 5.0. The unreacted materials left behind without reacting are collected and are recycled for the aromatization reaction.

Lower hydrocarbons that can be used as starting materials for producing an aromatic hydrocarbon for the purpose of the present invention include saturated and unsaturated hydrocarbons having 1 to 8 carbon atoms in a molecule that are gas in the conditions of the reaction. Specific examples of such hydrocarbons include methane, ethane, ethylene, propane, propylene, n-butane, isobutane, n-butene, isobutene, pentane, pentene, hexane, hexene, heptane, heptene, octane and octene, which may be straight chains or have a branched or ring structure.

Such hydrocarbons may be used alone, as a mixture or a product of any of various chemical processes. Examples of such gaseous product include petroleum gas, coal carbonization gas, refinery gas, naphtha, organic matter fermentation gas, organic matter dry distilled gas, coal reformed gas and methane hydrate recycling gas.

With a process for producing an aromatic compound according to the present invention, the carbon deposition on the surface of the catalyst can be suppressed by adding hydrogen, carbon monoxide, carbon dioxide and/or steam by 1 to 20 volume portions, preferably by 3 to 10 volume portions relative to 100 volume potions of the lower hydrocarbon in terms of volume ratio.

A process for producing an aromatic hydrocarbon according to the present invention is characterized by producing hydrogen as byproduct in addition to aromatic hydrocarbons mainly containing benzene.

Generally, with a process for producing hydrogen involving utilization of reforming vapor of lower hydrocarbons such as methane and a subsequent use of a shift converter, all the carbon in the lower hydrocarbons turns to carbon dioxide and the produced carbon dioxide is discharged into the atmosphere to raise the load of the environment.

To the contrary, with a process according to the present invention, all the carbon in the lower hydrocarbons are utilized to produce aromatic hydrocarbons. Thus, the present invention is advantageous from the viewpoint of environmental problems when the hydrogen produced as byproduct is used for fuel cells.

Now, the present invention will be described further by way of examples and comparative examples.

### EXAMPLE 1

(Preparation of Samples 1 through 4)

10 g of HZSM-5 showing a silica/alumina ratio of 32 and a specific surface area of 320 m²/g was used as each of the metallosilicate carriers of the four samples. Ethanol solutions respectively containing 37 mg, 92 mg 184 mg and 368 mg of 3-aminopropyltriethoxysilane were added to the respective metallosilicate carriers so as to be sufficiently adsorbed and carried by the carriers. Then, the carriers were dried at 120° C. for 16 hours and baked at 550° C. in the atmosphere for 4 hours to obtain four silicon-oxide-modified HZSM-5 carriers that were modified to respective amounts of 0.1 weight portions, 0.25 weight portions, 0.5 weight portions and 1.0 weight portions relative to 100 weight portions of metallosilicate in terms of silicon oxide.

Each of the HZSM-5 carriers that were obtained with different amounts of modification was impregnated with an aqueous solution prepared by dissolving 1.174 g of ammonium molybdate in 17 ml of ion exchange water and baked at 550° C. for 10 hours. As a result, Samples 1 through 4 were obtained with respective amounts of silicon oxide modification of 37 mg, 92 mg, 184 mg and 368 mg.

(Aromatic Hydrocarbon Production Test)

An aromatic hydrocarbon production test was conducted by using 1.2 g of each of the catalysts to produce an aromatic hydrocarbon, using gas containing methane and hydrogen as starting material, and confirm the performance of the catalyst.

In the production test, the catalyst was filled on a fixed bed and its temperature was raised to 550° C. in an airflow for 1 hour. Subsequently, the lower hydrocarbon starting material containing methane by 93 volume % and hydrogen by 7 volume % was supplied and the temperature was raised to 650° C., which temperature was maintained for 1 hour. Thereafter, the temperature was raised further to 750° C. and mixture gas was supplied under pressure of 0.3 MPa at a weight hourly space velocity (WHSV) of 2,700 ml/g/h.

The hydrogen and the methane in the reaction product were measured by gas chromatography, using a thermal conductivity detector, while the hydrocarbon in the reaction product was measured by gas chromatography, using a hydrogen flame detector.

The amount of reaction product obtained in 1 second per 1 g of the catalyst, or the number of n mols of hydrogen and aromatic hydrocarbon, was used as index of performance.

Some of the results obtained by the measurement are shown in Table 1.

### EXAMPLE 2

(Preparation of Samples 5 through 7)

The preparation process of Example 1 was followed except that 3-aminopropyltriethoxysilane used for modifying the metallosilicate carriers was replaced by silicon-containing substances having different compositions, which substances were used with different amounts for the respective samples of this example. More specifically, 175 mg of 3-aminopropyltrimethoxysilane was used to prepare Sample 5 showing an amount of modification of 1.1 weight portions in terms of silicon oxide relative to 100 weight portions of the metallosilicate carrier and 180 mg of propyltriethoxysilane was used to prepare Sample 6 showing an amount of modification of 1.1 weight portions in terms of silicon oxide relative to 100 weight portions of the metallosilicate carrier, while 375 mg of tripheylaminosilane was used to prepare Sample 7 showing an amount of modification of 1.1 weight portions in terms of silicon oxide relative to 100 weight portions of the metallosilicate carrier.

(Aromatic Hydrocarbon Production Test)

An aromatic hydrocarbon production test was conducted as in Example 1 by using 1.2 g of each of the catalysts to produce an aromatic hydrocarbon and confirm the performance of the catalyst.

Some of the results obtained by the measurement are shown in Table 1.

### EXAMPLE 3

(Preparation of Samples 8 and 9)

The preparation process of Example 1 was followed except that 3-aminopropyltriethoxysilane used for modifying the metallosilicate carriers was replaced by an ethanol solution containing 280 mg of 18-crown-6 sodium salt, or 275 mg of hexafluoropentanedion sodium salt to prepare catalysts of Sample 8 and Sample 9, each showing an amount of modification of 1.1 weight portions in terms of sodium oxide (Na₂O) relative to 100 weight portions of metallosilicate.

(Aromatic Hydrocarbon Production Test)

An aromatic hydrocarbon production test was conducted as in Example 1 by using 1.2 g of each of the catalysts to produce an aromatic hydrocarbon and confirm the performance of the catalyst.

Some of the results obtained by the measurement are shown in Table 1.

### EXAMPLE 4

(Preparation of Sample 10)

The preparation process of Example 1 was followed except that 3-aminopropyltriethoxysilane used for modifying the metallosilicate carriers was replaced by 230 mg of calcium acetylacetonate to prepare Sample 10, showing an amount of modification of 1.1 weight portions in terms of calcium oxide (CaO) relative to 100 weight portions of metallosilicate.

(Aromatic Hydrocarbon Production Test)

An aromatic hydrocarbon production test was conducted as in Example 1 by using 1.2 g of the catalyst to produce an aromatic hydrocarbon and confirm the performance of the catalyst.

Some of the results obtained by the measurement are shown in Table 1.

### COMPARATIVE EXAMPLE 1

(Preparation of Comparative Sample 1)

10 g of HZSM-5 was impregnated with an aqueous solution prepared by dissolving 1.17 g of ammonium molybdate in 17 ml of water and baked at 550° C. for10 hours to obtain a molybdenum-carrying HZSM-5 catalyst as Comparative Sample 1.

(Aromatic Hydrocarbon Production Test)

An aromatic hydrocarbon production test was conducted as in Example 1 by using 1.2 g of the catalyst to produce an aromatic hydrocarbon and confirm the performance of the catalyst.

Some of the results obtained by the measurement are shown in Table 1.

TABLE 1

| | reaction | | production rate (nmol/s/g) | | | |
|---|---|---|---|---|---|---|
| catalyst | time | | hydrogen | benzene | toluene | naphthalene |
| Sample 1 | 3 | hours | 3876 | 1537 | 93 | 284 |
| Mo/SiHZSM-5 | 8 | hours | 3947 | 1475 | 97 | 201 |
| | 24 | hours | 3835 | 1307 | 70 | 156 |
| Sample 2 | 3 | hours | 4516 | 1598 | 108 | 165 |
| Mo/SiHZSM-5 | 8 | hours | 4365 | 1630 | 99 | 112 |
| | 24 | hours | 4120 | 1562 | 102 | 76 |
| Sample 3 | 3 | hours | 4729 | 1541 | 89 | 10 |
| Mo/SiHZSM-5 | 8 | hours | 4510 | 1485 | 67 | 9 |
| | 24 | hours | 4369 | 1385 | 54 | 7 |
| Sample 4 | 3 | hours | 3867 | 1260 | 38 | 10 |
| Mo/SiHZSM-5 | 8 | hours | 3689 | 1154 | 43 | 7 |
| | 24 | hours | 3487 | 898 | 35 | 12 |
| Sample 5 | 3 | hours | 4024 | 1744 | 64 | 5 |
| Mo/SiHZSM-5 | 8 | hours | 3897 | 1764 | 55 | 8 |
| | 24 | hours | 3571 | 1690 | 29 | 10 |
| Sample 6 | 3 | hours | 4785 | 1580 | 78 | 158 |
| Mo/SiHZSM-5 | 8 | hours | 4853 | 1479 | 65 | 215 |
| | 24 | hours | 2570 | 857 | 38 | 132 |
| Sample 7 | 3 | hours | 3965 | 1780 | 189 | 87 |
| Mo/SiHZSM-5 | 8 | hours | 4512 | 1686 | 135 | 27 |
| | 24 | hours | 3260 | 875 | 82 | 11 |
| Sample 8 | 3 | hours | 3896 | 1787 | 75 | 24 |
| Mo/NaZSM-5 | 8 | hours | 3988 | 1686 | 96 | 20 |
| | 24 | hours | 3625 | 1767 | 75 | 8 |
| Sample 9 | 3 | hours | 3629 | 1719 | 120 | 5 |
| Mo/NaZSM-5 | 8 | hours | 3575 | 1678 | 125 | 10 |
| | 24 | hours | 3420 | 1659 | 84 | 8 |
| Sample 10 | 3 | hours | 3528 | 1986 | 45 | 13 |
| Mo/CaZSM-5 | 8 | hours | 3287 | 1785 | 195 | 10 |
| | 24 | hours | 2750 | 1635 | 115 | 8 |
| Comparative | 3 | hours | 4851 | 1259 | 105 | 553 |
| Sample 1 | 8 | hours | 4682 | 1286 | 90 | 413 |
| Mo/HZSM-5 | 24 | hours | 4444 | 1126 | 90 | 234 |

### EXAMPLE 5

(Preparation of Sample 11)

The preparation process of Example 1 was followed except that 3-aminopropyltriethoxysilane used for modifying the metallosilicate carriers was replaced by 475 mg of 3-aminopropyltrimethoxysilane to prepare modified metallosilicate, showing an amount of modification of 1.1 weight portions in terms of silicon oxide (SiO₂) relative to 100 weight portions of metallosilicate.

Then, 1.5 g of the obtained modified metallosilicate was impregnated with an aqueous solution prepared by dissolving 1.25 g of ammonium pararhenate in 20 ml of ion exchange water and then baked at 350° C. for 10 hours to obtain a silicon-oxide-modified HZSM-5 catalyst carrying rhenium oxide as Sample 11.

(Aromatic Hydrocarbon Production Test)

An aromatic hydrocarbon production test was conducted as in Example 1 by using 1.2 g of the catalyst to produce an aromatic hydrocarbon and confirm the performance of the catalyst.

Some of the results obtained by the measurement are shown in Table 2.

### EXAMPLE 6

(Preparation of Sample 12)

The preparation process of Example 1 was followed except that 3-aminopropyltriethoxysilane used for modifying the metallosilicate carriers was replaced by 426 mg of 3-trimethoxysilylpropyldiethylenetriamine to prepare modified metallosilicate, showing an amount of modification of 1.1 weight portions in terms of silicon oxide (SiO₂) relative to 100 weight portions of metallosilicate.

Then, 1.5 g of the obtained modified metallosilicate was impregnated with an aqueous solution prepared by dissolving 1.25 g of ammonium pararhenate and 0.275 g of chloropltinic acid in 20 ml of ion exchange water and then baked at 350° C. for 10 hours to obtain a silicon-oxide-modified HZSM-5 catalyst carrying rhenium oxide-platinum as Sample 12.

(Aromatic Hydrocarbon Production Test)

An aromatic hydrocarbon production test was conducted as in Example 1 by using 1.2 g of the catalyst to produce an aromatic hydrocarbon and confirm the performance of the catalyst.

Some of the results obtained by the measurement are shown in Table 2.

### EXAMPLE 7

(Preparation of Sample 13)

10 g of HZSM-11 was used as metallosilicate carrier, which was sufficiently impregnated with an ethanol solution containing 435 mg of 3-methylpropyltriethoxysilane and N-phenylaminopropyltriethoxysilane, then dried at120° C. for 16 hours and baked at 550° C. for 4 hours to obtain modified metallosilicate, showing an amount of modification of 1.0 weight portions in terms of silicon oxide (SiO₂) relative to 100 weight portions of metallosilicate.

Then, 5 g of the obtained modified metallosilicate was impregnated with an aqueous solution prepared by dissolving 1.25 g of ammonium molybdate and 0.35 g of rhodium chloride in 25 ml of water and then baked at 350° C. for 10 hours in the atmosphere to obtain a silicon-oxide-modified ZSM-11 catalyst carrying Mo—Rh as Sample 13.

(Aromatic Hydrocarbon Production Test)

An aromatic hydrocarbon production test was conducted as in Example 1 by using 1.2 g of the catalyst to produce an aromatic hydrocarbon and confirm the performance of the catalyst.

Some of the results obtained by the measurement are shown in Table 2.

### EXAMPLE 8

(Preparation of Sample 14)

The preparation process of Example 7 was followed except that 10 g of MCM-22 was used as metallosilicate carrier to prepare modified metallosilicate, showing an amount of modification of 1.0 weight portions in terms of silicon oxide (SiO₂) relative to 100 weight portions of metallosilicate.

Then, 5 g of the obtained modified metallosilicate was made to carry catalyst metal components as in Example 7 to obtain a silicon-oxide-modified MCM-22 catalyst carrying Mo—Rh as Sample 14.

(Aromatic Hydrocarbon Production Test)

An aromatic hydrocarbon production test was conducted as in Example 1 by using 1.2 g of the catalyst to produce an aromatic hydrocarbon and confirm the performance of the catalyst.

Some of the results obtained by the measurement are shown in Table 2.

### EXAMPLE 9

(Preparation of Sample 15)

The preparation process of Example 7 was followed except that 10 g of β-zeolite was used as metallosilicate carrier to prepare modified metallosilicate, showing an amount of modification of 1.0 weight portions in terms of silicon oxide (SiO₂) relative to 100 weight portions of metallosilicate.

Then, 5 g of the obtained modified metallosilicate was impregnated with an aqueous solution prepared by dissolving 1.25 g of ammonium molybdate and 0.52 g of chloroplatinic acid in 25 ml of water and then baked at 350° C. for 10 hours to obtain a silicon-oxide-modified zeolite catalyst carrying Mo—Pt as Sample 15.

(Aromatic Hydrocarbon Production Test)

An aromatic hydrocarbon production test was conducted as in Example 1 by using 1.2 g of the catalyst to produce an aromatic hydrocarbon and confirm the performance of the catalyst.

Some of the results obtained by the measurement are shown in Table 2.

### COMPARATIVE EXAMPLE 2

(Preparation of Comparative Sample 2)

10 g of HZSM-5 was impregnated with an aqueous solution prepared by dissolving 1.24 g of ammonium pararhenate in 17 ml of water and then baked at550° C. for 10 hours to obtain an HZSM-5 catalyst carrying rhenium as Comparative Sample 2.

(Aromatic Hydrocarbon Production Test)

An aromatic hydrocarbon production test was conducted as in Example 1 by using 1.2 g of the catalyst to produce an aromatic hydrocarbon and confirm the performance of the catalyst.

Some of the results obtained by the measurement are shown in Table 2.

TABLE 2

| | reaction | | production rate (nmol/s/g) | | | |
|---|---|---|---|---|---|---|
| catalyst | time | | hydrogen | benzene | toluene | naphthalene |
| Sample 11 | 3 | hours | 4125 | 1864 | 84 | 18 |
| Re/SiHZSM-5 | 8 | hours | 3895 | 1760 | 60 | 13 |
| | 24 | hours | 3589 | 1693 | 89 | 8 |
| Sample 12 | 3 | hours | 3266 | 1566 | 67 | 20 |
| Re—Pt/ | 8 | hours | 3796 | 1498 | 55 | 12 |
| SiHZSM-5 | 24 | hours | 2870 | 1250 | 48 | 9 |
| Sample 13 | 3 | hours | 3846 | 1736 | 74 | 54 |
| Mo—Rh/ | 8 | hours | 3950 | 1685 | 87 | 25 |
| Si β-zeolite | 24 | hours | 3725 | 1767 | 75 | 18 |
| Sample 14 | 3 | hours | 3629 | 1749 | 129 | 9 |
| Mo—Rh/ | 8 | hours | 3615 | 1695 | 135 | 12 |
| SiZSM-11 | 24 | hours | 3420 | 1659 | 84 | 5 |
| Sample 15 | 3 | hours | 3522 | 1986 | 45 | 18 |
| Mo—Pt/ | 8 | hours | 3287 | 1835 | 195 | 12 |
| SiMCM-22 | 24 | hours | 2750 | 1587 | 120 | 8 |
| Comparative | 3 | hours | 4851 | 1369 | 105 | 583 |
| Sample 2 | 8 | hours | 4682 | 1485 | 94 | 523 |
| Re/HZSM-5 | 24 | hours | 3245 | 1016 | 75 | 265 |

### INDUSTRIAL APPLICABILITY

A process for producing an aromatic hydrocarbon according to the present invention employs a catalyst prepared by causing a metallosilicate carrier that is modified by means of a compound containing silicon, alkali metal or alkaline-earth metal to carry the metal or oxide thereof. Therefore, it is possible to produce aromatic hydrocarbons containing benzene as main component with a high selectivity and a high yield from the initial stages of reaction, using lower hydrocarbons and the yield does not decrease with reaction time. Thus, the present invention provides a highly efficient process for producing an aromatic hydrocarbon by using lower hydrocarbons as starting material.

1. A process for producing an aromatic hydrocarbon, using a lower hydrocarbon as starting material, the process comprising heating the hydrocarbon in the presence of a catalyst carrying a molybdenum compound or a rhenium compound on a metallosilicate carrier modified with a silicon compound, a sodium compound or a calcium compound, wherein the silicon compound is a silane compound having a basic group selected from aminopropyl and pyridyl groups and an organic group of a size equal to or greater than the pore size of the metallosilicate and selected from trialkoxy and triphenyl groups and the sodium compound or the calcium compound is a compound having an organic group of a size equal to or greater than the pore size of the metallosilicate and selected from crown ether, hexafluoropentanedione and acetylacetonate, and said metallosilicate carrier being modified so as to make an oxide thereof by impregnating said silane compounds, said sodium compound or said calcium compound the metallosilicate carrier with it and subsequently heat-treating it in an oxygen-containing atmosphere.

2. The process according to claim 1, wherein the catalyst contains platinum or rhodium.

3. A catalyst for producing an aromatic hydrocarbon, using a lower hydrocarbon as starting material, the catalyst carrying a molybdenum compound or a rhenium compound on a metallosilicate carrier modified with a silicon compound, a sodium compound or a calcium compound, wherein the silicon compound is a silane compound having a basic group selected from aminopropyl and pyridyl groups and an organic group of a size equal to or greater than the pore size of the metallosilicate and selected from trialkoxy and triphenyl groups and the sodium compound or the calcium compound is a compound having an organic group of a size equal to or greater than the pore size of the metallosilicate and selected from crown ether, hexafluoropentanedione and acetylacetonate, and said metallosilicate carrier being modified so as to make an oxide thereof by impregnating said silane compounds, said sodium compound or said calcium compound the metallosilicate carrier with it and subsequently heat-treating it in an oxygen-containing atmosphere.

4. The catalyst according to claim 3, wherein carrying platinum or rhodium compound in addition to the molybdenum compound or the rhenium compound.
